(19) United States
(12) Patent Application Publication
(10) Pub. No.: US 2023/0029969 A1
(43) Pub. Date: Feb. 2, 2023

(54) Orthodontic Chew, Comfort Tape, and Therapeutic Sticker for Use in the Mouth

(71) Applicant: ORTHONU, LLC, Rumson, NJ (US)

(72) Inventors: Sima Yakoby Epstein, Rumson, NJ (US); Charles E. Lundy, JR., Raleigh, NC (US); Harold Howlett, Gulfport, MS (US)

(21) Appl. No.: 17/958,294

(22) Filed: Sep. 30, 2022

Related U.S. Application Data
(63) Continuation-in-part of application No. 17/581,825, filed on Jan. 21, 2022.
(60) Provisional application No. 63/140,247, filed on Jan. 21, 2021.

(51) Int. Cl. A61C 7/12

(52) U.S. Cl. CPC A61C 7/125

(57) ABSTRACT

A tape for use with braces or aligners includes a member of flexible and stretchable material that is infused with an ingredient to improve oral care during treatment with braces or aligners. The material includes two or more layers of material that are laminated together and that include a first layer of polymer film and a second layer of hydrogel material.

### CROSS-REFERENCE TO RELATED APPLICATION

This application claims priority to U.S. Provisional Patent Application No. 63/140,247, filed Jan. 21, 2021, and to U.S. Utility patent application Ser. No. 17/581,825, filed Jan. 21, 2022, the disclosures of which are incorporated herein by reference in their entirety.

### FIELD

The invention concerns an orthodontic chew, comfort tape, and therapeutic sticker for use in orthodontia care inside the mouth.

### BACKGROUND

In orthodontic care of patients, there are at least three areas of concern that must be addressed, and which are currently under-addressed in consumer solution products. These include 1) emergency care, 2) oral hygiene, and 3) visual challenges. Emergency care is needed to address discomfort, pain, sensitivity, ulcerations, dry mouth, and the like.

Oral hygiene involves long term oral care and prevention. Oral hygiene includes maintenance care for preventing halitosis and preventing or treating gingivitis and the increase in gram negative bacteria on the tongue and periodontium that are associated with orthodontia care. Avoiding gingivitis and reducing gram negative bacteria in a patient's mouth can prevent and/or deter bone loss and inflammation. This can help to maintain a healthy mouth, tongue, gums, and fresh breath.

Visual challenges include the appearance of white decalcifications on teeth due to the start of cavities that can be attributed to an increase of gram-positive bacteria. Other visible challenges include broken brackets, tooth and elastic discoloration, food stuck between teeth, and staining of enamel.

Gram negative bacteria lives on the tongue and inside the gum line around the periodontium. Gram negative bacteria is a main cause of halitosis in the mouth. Gram positive bacteria is found in the plaque accumulation of the pellicle that forms on the enamel of the tooth that then starts the decalcification process that can lead to cavities, also causing halitosis. Targeting these two types of bacteria with different ingredients and tools and differentiating them can solve many issues inherent in the changes in microbiome during orthodontic treatment.

Patients undergoing orthodontic treatment often undergo multiple adjustments to their braces and/or aligners throughout their treatment. Patients oftentimes use homemade “fixes” when it is impossible to get to their Orthodontists Office. Some examples include eraser heads for broken wires and toenail clippers or wire clippers to cut wires. These options are often not the most hygienic choices and improvements can be made to help the orthodontic patient in dealing with these types of emergencies. Shifting teeth can also trigger increases in bacterial loads. Because of the lack of home care solutions, patients often simply live with the bad breath and plaque that builds up on their teeth. On-the-go solutions and home-care solutions are needed.

### SUMMARY

An orthodontic chew is shown and described. The chew may be shaped like a straw. Comfort tape for use with braces is also shown and described. A therapeutic sticker for use in the mouth is also shown and disclosed

### BRIEF DESCRIPTION OF THE DRAWINGS

FIG. 1 depicts a perspective view of a roll of comfort tape according to the invention, with the tape stored in a dispenser;

FIG. 2 depicts a plan view of an alternative dispenser for a comfort tape strip;

FIG. 3 depicts a plan view of an alternative dispenser for a comfort tape strip stored in a case with a cover;

FIG. 4 depicts a perspective view of an alternative dispenser for a comfort tape strip stored in a case with a lid;

FIG. 5 depicts a top, bottom, and side plan view of a chew according to the invention;

FIG. 6 depicts a perspective view of an alternative design for the chew where the chew is curved;

FIG. 7 depicts a side plan view of the chew of FIG. 6;

FIG. 8 depicts a top plan view of the chew of FIG. 6;

FIG. 9 depicts a bottom plan view of the chew of FIG. 6;

FIG. 10 depicts an end plan view of the chew of FIG. 6;

FIG. 11 depicts a perspective view of an alternative chew according to the invention, with the chew being straw-shaped;

FIG. 11 depicts an exploded view of the chew of FIG. 11; and

FIG. 12 depicts a perspective view of an alternative embodiment of a chew that is straw shaped and has a compartment for dispensing a fluid;

FIG. 13 depicts a front plan view of an inventive dental floss for use in flossing teeth with braces;

FIG. 14 depicts a front plan view of an inventive dental pick;

FIG. 15 depicts a front plan view of another inventive dental pick;

FIG. 16 depicts a top plan view of a comfort strip or tape according to the invention for application to braces and/or teeth of a patient;

FIG. 17 depicts a bottom plan view of the comfort tape or strip of FIG. 16;

FIG. 18 depicts a top plan view of a therapeutic sticker according to the invention for application to the soft tissue in a patient's mouth;

FIG. 19 depicts a bottom plan view of the sticker of claim 18;

FIG. 20 depicts a cross-sectional exploded side view of an example comfort tape or strip according to the invention; and

FIG. 21 depicts a carrier card having multiple comfort tapes or strips according to the invention.

### DETAILED DESCRIPTION

Science has shown that most of the bacteria in a patient's mouth resides on the tongue. During orthodontic treatment, patients have challenges with oral hygiene. As teeth shift, the mouth has a greater propensity to harbor bacteria in both the gums and on the tongue. In addition, teeth can be damaged due to demineralization and a patient may experience pain during the treatment process.

The present invention concerns various orthodontic treatment vehicles or elements that can be placed in the mouth and used by a user to assist in oral care. For example, the present invention concerns vehicles such as functional candies (e.g., lozenges and lollipops); bon bons (edible chocolates); hydrogels (comfort strips or tapes); mucoadhesive buccal films; gels, pastes, or sprays; or mouth based ingestibles (chews). These elements are used in combating four categories of paint points in orthodontics, including: 1) paint, discomfort, and inflammation treatment, 2) oral hygiene, halitosis, and anti-bacterial treatment, 3) demineralization, plaque, staining, and anti-microbial treatment, and 4) dry mouth and acidic saliva PH.

Various ingredients are believed to be useful in the treatment vehicles for different purposes. These ingredients include:

1) Carica papaya extract and seed (oil and other forms) for treating plaque-induced gingivitis, periodontitis, and oral malodor. There is a proportionately high amount of tannins in the seeds of C. papaya which explains its high antimicrobial activity. 10% Carica papaya L. seed extract can reduce dental plaque score in patients with gingivitis. 2.5% papaya leaf extract solution can decrease the plaque index and gingival index in moderate gingivitis.

2) Clove oil extract is an antioxidant. antifungal, and antibacterial. 4.7% clove gel provides very strong radical scavenging activity, possesses antifungal activity, and possesses an inhibitory effect on multi-resistant Staphylococcus spp.

3) Lemon oil extract is an antifungal that contains almost exclusively terpenes and oxygenated terpenes. It possesses antifungal potential against three Candida species (C. albicans, Candida tropicalis, and Candida glabrata). Lemon oil extract may also be an effective remedy against candidiasis caused by C. albicans.

4) Green tea extract can significantly reduce oral volatile sulfur compound (VSC) levels. 5) Wasabi extract can inhibit growth of bacteria, is antimicrobial, helps to reduce inflammation and can reduce pain. It also may have anticancer effects. Wasabi extract contains phytonutrients or phytochemicals, called isothiocyanates. Isothiocyanates are sulphur-containing phytonutrients with strong anticancer properties. Isothiocyanates inhibit the growth of Streptococcus mutans, which is the bacteria that causes dental caries. These isothiocyanates can interfere with the sugar-dependent adherence of the cells to the teeth. Isothiocyanates may block inflammation which, in turn, could provide pain killing properties.

6) Ginger extract has been validated for oral care. Ginger extracts possess antimicrobial, antifungal, oral anticancer, anti-nausea, anti-carries, and antiplaque properties. Ginger also hardens the teeth due to indirect mineralization properties. Overall, the health benefits of ginger in oral care make it useful for treatment of various oral disorders.

7) Cocoa extract in the form of Cacao bean husk ethanol extract (CBHEE) has polyphenol that acts as an antibacterial agent.

8) Rose extract can provide relief from inflammation and gum problems. It can also strengthen teeth and provide relief from gum pain. If you are suffering with bad odor and foul breath, rose water/extract can help to eliminate it.

9) Coconut oil extract provides substantial antimicrobial activity that is attributed to the presence of monolaurin in coconut oil. Coconut oil extract provides antimicrobial activity against Escherichia vulneris, Enterobacter spp., Helicobacter pylori, Staphylococcus aureus, Candida spp., including C. albicans, C. glabrata, C. tropicalis, C. parapsilosis, C. stellatoidea, C. krusei, S. mutans, and C. albicans.

10) Vanilla extract provides antioxidant activity. At a concentration of 200 ppm, vanilla extract shows 26% to 43% of antioxidant activity.

11) Dead sea salt is antibacterial, provides plaque control, and promotes oral wound healing. Sea salt along with xylitol and lysozyme was tested in a novel mouth rinse in adolescent patients aged 1417 years old. The study revealed that the use of sea salt-based mouthwash in daily oral hygiene reduced bacterial levels of S. mutans (p<0.01) with the combined action of xylitol and lysozyme. A 1.8% NaCl solution provided the strongest effect in reducing wound area size both at 24 and 48 hours. Thus, the use of sea salt as a mouth-rinse in conjunction with routine oral care has been shown to promote oral would healing.

12) Xylitol reduces the risk of caries in young patients wearing fixed orthodontic appliances. Regular use of polyol gum, especially polyol gum that contains xylitol as the prominent sweetener has been shown to reduce the risk of caries in young patients wearing fixed orthodontic appliances.

13) Monk fruit extract or liquid provides antioxidant effects and anti-inflammatory properties. The antioxidant effects of mogrosides have been shown to reduce DNA damage by free radicals, which scientists associate with cancer growth. The mogrosides in monk fruit provide antioxidant characteristics that protect cells from damage caused by free radicals. Damage caused by free radicals is a primary source of inflammation, such that monk fruit provides anti-inflammatory properties.

14) Miswak extract helps to prevent caries and reduce inflammation. Rinsing with miswak extract has been shown to stimulate parotid gland secretion (p<0.01) and raise the plaque pH. This suggests that Miswak extract may prevent caries formation. Miswak extract can affect dental plaque and gingival inflammation scores.

15) Zinc acetate may favorably impact enamel demineralization and dental hypersensitivity.

16) Mint extract or oil derived from peppermint is known for its cooling and numbing effects. This can sooth tooth and muscle aches. Peppermint oil is powerful for fighting oral pathogens and killing common bacteria that leads to cavities and gum disease.

17) Probiotic, L. salivarius WB21 reduces halitosis and reduces bleeding. 2.0×10(9) Lactobacillus salivarius WB21 and xylitol administered orally has been shown to improve physiologic halitosis and has also been shown to provide beneficial effects on bleeding when probing from the periodontal pocket.

18) Calcium carbonate provides enamel remineralization enhancement. A CaCO(3)-based dentifrice has been shown to be more effective than a negative control on the enhancement of enamel remineralization.

19) Sodium bicarbonate provides salivary PH increase, restricts enamel demineralization, and reduces dental hypersensitivity. 3 g of sodium bicarbonate with 50 mill of water produced a significant salivary PH increase and restricted enamel demineralization. The use of sodium bicarbonate can support the buffering capacity of saliva to help in caries prevention, dental erosions, treatment of xerostomia, and treatment of dental hypersensitivity.

20) Magnolia bark extract provides halitosis management. Gum that contains magnolia bark extract (in the amount of 0.15% by weight) along with zinc acetate (in the amount of 0.012% by weight) has been shown to reduce oral VSC levels for more than one hour. Chewing gums containing probiotics Lactobacillus, zinc acetate and magnolia bark extra, eucalyptus extract, and AITC with zinc lactate may be suitable for halitosis management.

21) Chitosan provides antibacterial and antimicrobial properties. Chitosan can be used in different forms, such as solution, powder, flake, fiber, and film. Chitosan contained in chewing bum has a greater antibacterial effect and increases salivary secretion, which is desirable in improving general oral hygiene and in preventing progression of periodontal disease. In addition, chitosan provides potent oral antimicrobial activity.

22) L-arginine destabilizes and removes mature oral biofilms and relieves sensitivity. Acidic L-arginine (0.5 M, pH 3.5) prepared with citrate buffer (pH 3.5) can be used as an additive that enhances the efficacy of mouth rinses used in oral hygiene. L-arginine (8%) along with calcium carbonate and phosphate in the form of a plug has been shown to seal dentin tubules. This plus has been shown to be resistant to normal pulpal pressures and to acid challenge. This effectively reduces dentin fluid flow and helps to relieve sensitivity.

23) Guava leaf extract provides anti-plaque, anti-microbial, anti-inflammatory and antioxidant properties. Guava leaf extract has been shown to be a potent anti-inflammatory agent as an oral therapeutic mouth rinse. Guava leaf extract has also been shown to be effective in treatment of periodontal disease. Guava leaves also help to maintain strong teeth and gums.

24) Eucalyptus extract and oil provides anticariogenic agent. Eucalyptus extract/oil shows an inhibitory effect on oral pathogens like Lactobacillus acidophilus, making it suitable as an anticariogenic agent.

25) P. betle leaves may be used in different forms, such as extract, oil, or other forms. P. betle leaves display high efficiency on Gram-negative bacteria, such as Escherichia coli and Pseudomonas aeruginosa, and on Gram-positive bacterial, such as Staphylococcus aureus, and Candida albicans.

26) Selectivity Filter (sf)—potassium ions treat tooth sensitivity. The minimum concentration needed to sustain nerve depolarization in teeth has been shown to be 0.08%.

27) Hydro-alcoholic polyphenolic extracts of Robinia pseudoacacia (acacia honey), Myrtus communis leaves, and Punica granatum L. fruit peel have been shown to counteract cariogenic bacteria of dental plaque.

There are likely other ingredients that can also be used in reducing bacterial levels in the mouth, reducing the likelihood of caries generation, are anti-inflammatory, anti-microbial, antioxidants, and are likely to treat halitosis. Other ingredients may alternatively be used as desired in the various treatment vehicles.

At present, there are no functional candies that are designed specifically for use with braces. Some orthodontists allow their patients to chew sugar free gum, but patients are told to avoid sticky candies and taffies of all types, for fear that use of such products may result in damage to brackets and wires.

Most braces wearers are children and teens, and snacks are an integral part to their lives. A functional orthodontic candy should taste great and be safe to eat during treatment. In addition, functional orthodontic candy can have added benefits, which may include: 1) numbing effects to alleviate soreness, 2) cavity prevention, and 3) cleaning features as you chew, such as removing food particles from teeth and braces. Orthodontic candies that taste great and are safe for use with braces and aligners will allow the patient to eat during treatment.

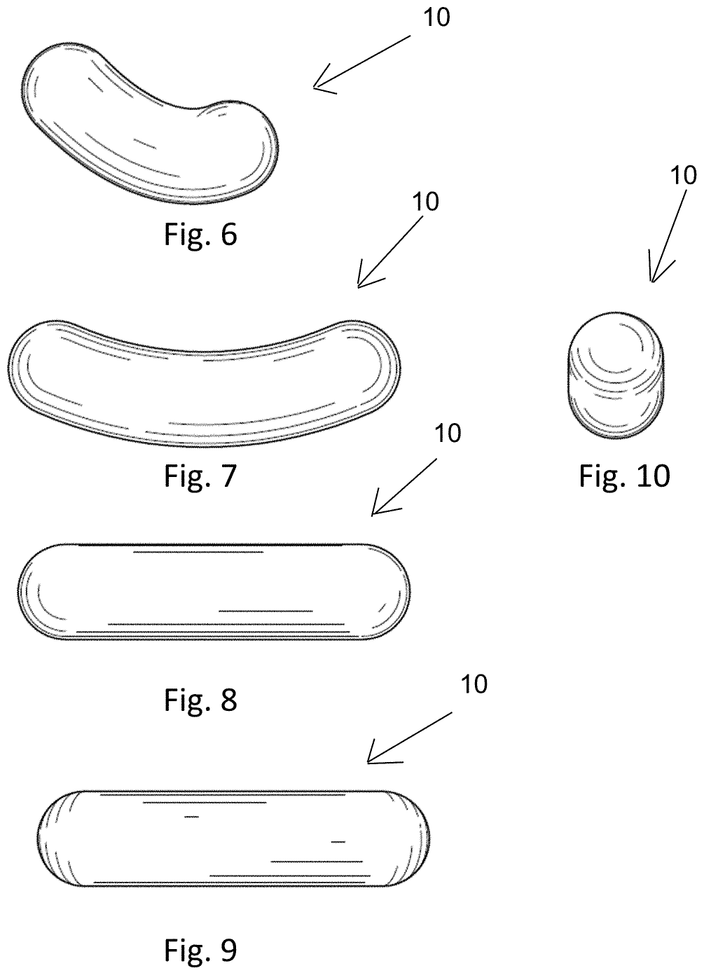

Referring to FIGS. 6-10, one type of functional candy is an “everlasting,” non-edible, and/or reusable chew 10. Some children have an oral fixation, which causes them to chew things like pens, erasers, and like. This can cause issues with braces, such as broken wires and brackets. Some people suffer from dry mouth. Chewing stimulates the salivary glands to produce saliva. A long-lasting chew product can help stimulate saliva to resolve dry mouth issues. Everlasting chews 10 can provide children who have an oral fixation with a “chew-safe” tool that has added health benefits.

Everlasting chews 10 may be shaped so that they are conducive to chewing, such as tubular or eraser shaped, and can be stored in a case between chews. A chew 10 of this nature may be used multiple times, for a set period, such as, for example, up to 16 hours. The product may include a ‘time-release’ solution that is safe to chew, has flavor benefits, and/or slowly releases a solvent with added benefits. This product can take the place of gum or candy. The chew 10 must be choke proof.

The “everlasting” chew 10 can have additional benefits beyond being chewable, including:

1) A baking soda blend with hydroxyapatite and whitening for cleaning, whitening, and plaque control to keep enamel mineralized;

2) A combination of mint, licorice, and xylitol for improving breath and producing saliva;

3) A combination of eucalyptus, menthol, and lidocaine for pain or discomfort issues; or

4) A combination of any of the foregoing ingredients or other ingredients.

This flavorful, chew-safe tool has added health benefits. It may come in a small case that stores multiple chews 10 at a time. The chews 10 may be small, flavorful, chewable food-grade silicone and may include slow-release flavors and functional benefits. One type of food-grade silicone is siloxane polymer having a hardness of Shore 00-35. Other types of materials and hardness may be used, if desired. The chew 10 may have a center matrix of natural polysaccharide or pvp base, xylitol, sodium bicarbonate, menthol, or spearmint oil.

Functional benefits may include, but are not limited to, numbing effects, cleaning the teeth, whitening and remineralizing the teeth, relieving the desire to chew, preventing mouth dryness, helping to seat an appliance (such as an aligner), or reducing halitosis.

The chews 10 may have an approximate size of 8 mm×10 mm×4 mm and may be packed as 12 pieces in a package. The chew 10 may have a curved shape, as shown in FIGS. 6-10, or a straight shape, as shown in FIG. 5. The chew 10 can have a round cross-sectional shape, or other cross-sectional shapes, such as square, oval, triangular, or other shapes. The chews 10 preferably have a long shelf-life, such as 2 years at minimum.

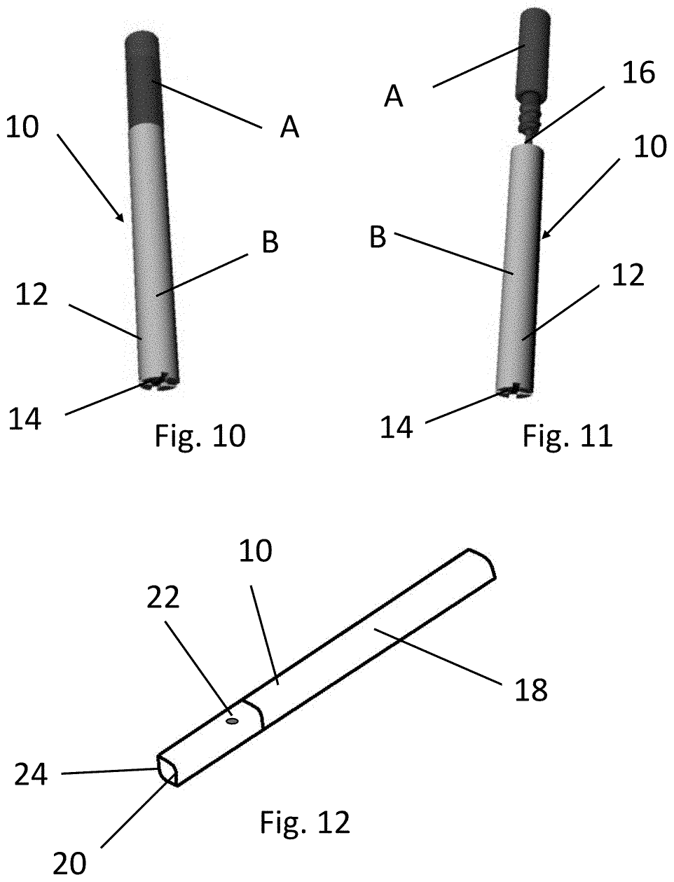

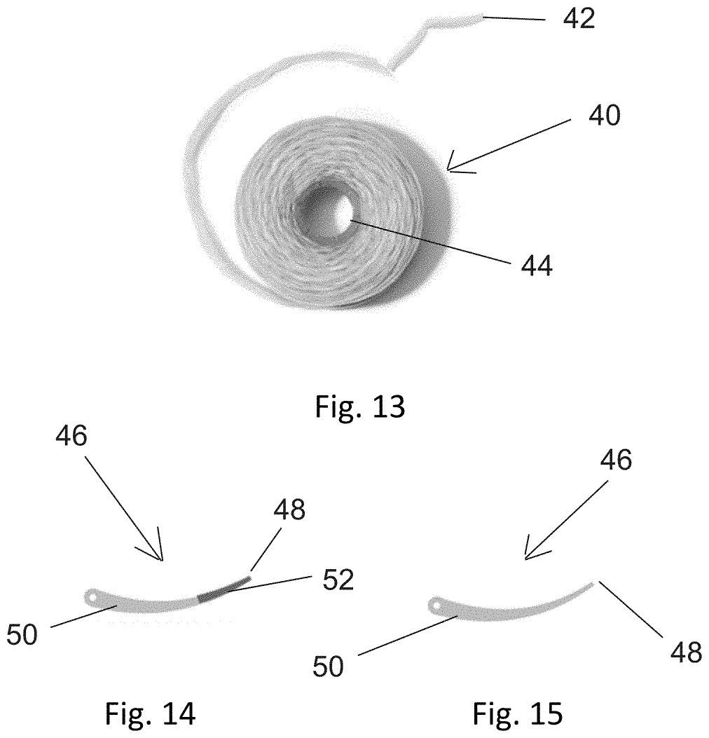

An alternative embodiment of the chew 10 is shown in FIGS. 10-12. In this embodiment, the chew 10 is elongated and straw shaped. The chew 10 is made of silicone. The silicone may include platinum-based catalysts. The silicone may have a Shore A 10 hardness or a similar hardness. The straw may be molded or extruded.

The straw is an elongated, straight member 12 with a hollow interior 14. The straw is tube-shaped along at least part of its length. The straw may come in multiple parts A, B or be a single part. When multiple parts are used, one end A of the straw may screw into or be press fit into (or otherwise attached to) the other part B. The straw is non-edible and long lasting such that a patient will find it to be fun to chew. In addition, the center of the straw may be filled with a dissolvable flavored core that has an appealing flavor to children, teens, and adults. A user chews on the straw to appease an oral fixation and desire to chew, or to help to seat an aligner or other appliance. The straw can also help to prevent mouth dryness and to reduce halitosis. In addition, the straw may be used for medicament delivery.

The core 14 may be filled with a dissolvable flavored core of xylitol, baking powder, an antimicrobial agent and coffee, berry, or other available flavors. Alternatively, the straw may be fillable with a liquid flavorant.

An example straw design is depicted in FIGS. 10-11, where the straw is made of two parts, with an upper part A being removable from a lower part B. The upper part A may screw into the lower part B and may include a guide arm 16 to guide the upper part into the inner open part 14 of the chew 10. Flavor drops may be dispensed into the center of the chew 10 by removing the upper part A, or cap, from the silicone chew 10.

An alternative embodiment is shown in FIG. 12. In this embodiment, the straw has a paper sleeve 18 at one end and a silicone chew 10 at the other end. The paper sleeve 18 is for holding the chew 10. Other types of material could alternatively be used for the sleeve, such as a plastic or thermoplastic material or other known materials. The silicone chew 10 has a tapered down portion 20 at one end that can be used by a patient for chewing purposes. The chew 10 includes an open interior surrounded by silicone. A hole 22 is provided on the side of the chew 10 for allowing the input of liquid drops into the chew 10. An exit hole 24 for the flavor drops is provided at the end of the silicone chew 10, where a user would chew on the chew 10.

Other shapes, designs, and sizes may be used for the straw, as will be evident from the teachings herein. Preferably, the silicone chew 10 is designed to permit a user to taste any flavorant that is applied and to receive any treatments or medicaments applied to the chew 10, whether on the interior, exterior, or imbedded in the material.

The chews 10 may come in any variety of flavors. Example flavors include lime, lemon, orange, cherry, licorice, bubble gum, grape, blueberry, cinnamon, chai, mocha latte, and coffee, among other flavors.

Another type of functional candy that is safe to chew is licorice root (with whitening baking soda) to remove external enamel stains and hydroxyapatite to re-mineralize enamel. Another type is homeopathic candies that may include peppermint that has a natural antiseptic property to help with bad breath and menthol mixed with lidocaine to create a soothing numbing effect for sensitivity and discomfort. Another combination may include chlorhexidine, sodium chlorite, manuka oil extract, parsley, mint, and cocoa extract to create an anti-inflammatory and antibacterial effect on periodontium as well as freshen breath.

The functional candies of the invention may also be edible, such as candy infused with functional benefits. One example is vanilla sugar cookie, which is a chocolate or bonbon infused with anti-inflammatory and anti-bacterial properties to fight gingivitis. Another example is mint chocolate chip, which is a chocolate or bonbon that may include a mint extract and whitening agents to eliminate bad breath and remove extrinsic stains on enamel. Another example is caramel latte, which is infused with pre and probiotics, vitamins and minerals promoting a healthy environment in one's mouth. Many other flavors or ingredients may be used.

Functional orthodontic candy, mints, and gum can also help to stop plaque accumulation on the teeth simply by chewing. The candies and gum can include any number of ingredients, such as chlorhexidine, lidocaine (for pain), baking soda (for whitening), hydroxyapatite (remineralization and kills bacteria and stops cavities), licorice root, sodium hydroxide, and a caramel sea salt rinse that the patient can swoosh around the mouth. Functional orthodontic candy, mints, and gum can also help to stop plaque accumulation on the teeth simply by chewing.

In practice, first, the chewing of the candy and gum massages around teeth, wires, brackets, rubber bands and gums. The specially formulated ortho candy preferably has no abrasiveness or stickiness, or limited stickiness. Natural ingredients may preferably be used, including peppermint, vitamins (D, C), orthodontic vitamins for healthy gum and teeth care, and xylitol, among other ingredients. The functional candy may include melt-in-your-mouth-chocolates, licorice, taffy, melts, cookies, mints, and gum, among other candies.

Lozenges may be provided to provide pain relief and may include as ingredients green tea menthol—90% PEG 1450, 3.8% Gum Arabic, 2% Citric Acid, 2% Stevia, 1% Menthol Crystals, 1% Lidocaine, and 0.2% Green Tea Extract. Another possible combination is a cinnamon cherry flavored lozenge that incorporates cinnamon oil and cherry flavoring, 86.9% PEG1450, 5% Gum Arabic, 3% Stevia, 2% Ascorbic Acid, 2% Cinnamon Oil Extract, 1% Lidocaine, 0.5% Ze Cherry Flavor, 0.5% Ze Bitter Blocker. Alternatively, in this combination, menthol crystals could be substituted for the lidocaine or could lower the amount of lidocaine to 0.25%. This combination could also be different flavors instead of cinnamon cherry, such as cherry without cinnamon oil, or blue raspberry. Alternatively, an embodiment could substitute a combination of menthol crystals and sodium chloride for the lidocaine.

Lozenges may be provided for treating halitosis and may include as ingredients green tea menthol—90% PEG 1450, 3.8% gum Arabic, 2% citric acid, 2% Stevia, and 0.2% green tea extract. Lozenges may also be provided to clean and remineralize the teeth. One possible flavor is strawberry and would include as ingredients 80.5% PEG 1450, 3% Gum Arabic, 3% Ascorbic Acid, 3% Sodium Bicarbonate, 3% Calcium, 3% Stevia, 2.5% Vitamin D, 1% Lidocaine, 1% Ze, and strawberry flavor. If desired, the lidocaine could be removed and substituted with phosphorous, xylitol and probiotic, L. salivarius WB21.

According to another aspect of the invention, tapes, or strips 30 for use with orthodontic treatment are disclosed. When a patient starts orthodontic treatment with braces, they often suffer from discomfort, pain, and sores. The brackets and attachments in clear aligners are rough and create sores in the vestibule, soft tissue/lip, and other places. Current palliative rinses can be used to soothe this discomfort, and small dots/waxes can also be positioned on brackets, wires, and the like. There are other products on the market, such as Orthodots' by Oral B, where you take off one piece at a time, hold for 30 seconds, and apply it to brackets. Often these small dots or pieces of wax fall off quickly and/or can be swallowed because they do not adhere in a lasting manner.

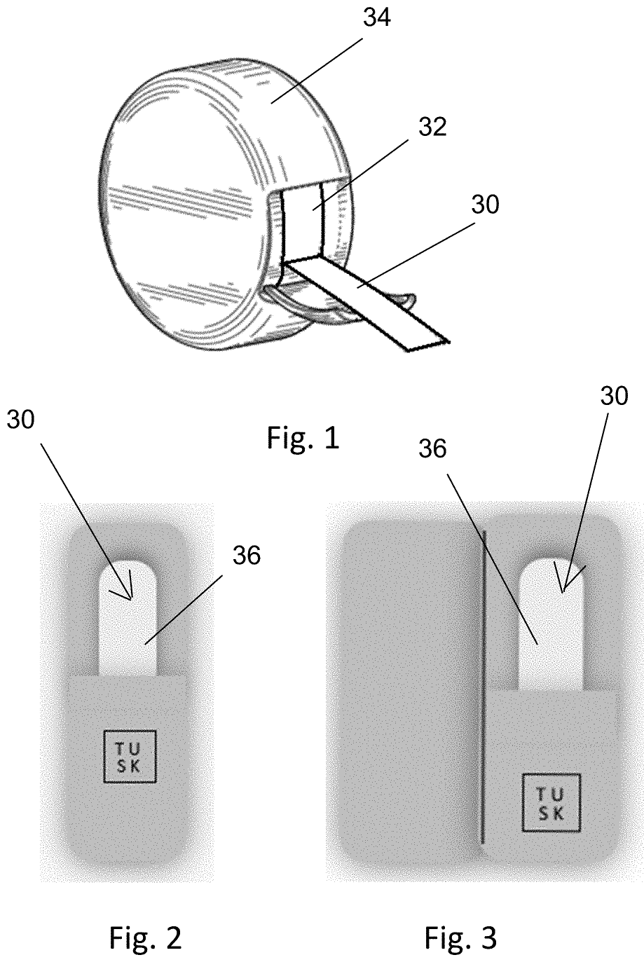

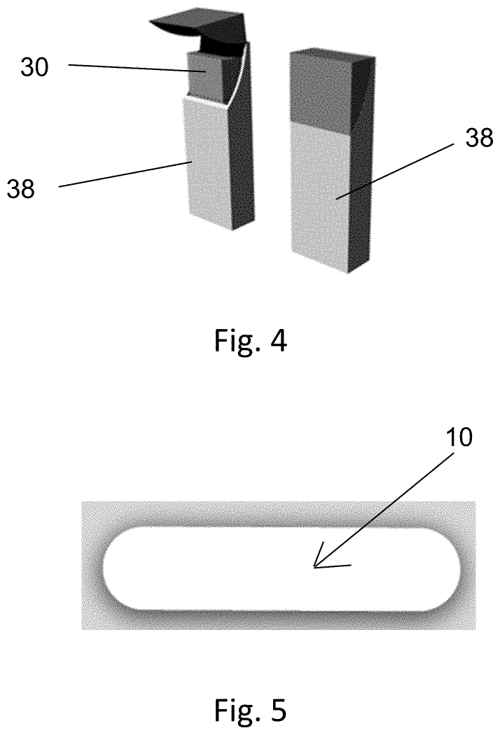

Referring to FIGS. 1-4, a bracket tape 30 according to the invention is different and includes a tape 30 and/or a roll that is stretchy and can be cut to cover the teeth adjacent the area of the mouth that is experiencing discomfort. The bracket tape 30 can be used to cover a larger area and can be made more secure than small dots or wax. Bracket tape 30 may be designed in silicone to be attached to brackets initially in the first weeks of treatments where most uncomfortable soreness develops to avoid ulcerations in the vestibule, lip, and cheek area of the mouth. Tape 30 of this nature stays on better because it has a cantilever effect, covering a bigger surface area adding retention of the tape to the brackets. In an alternative embodiment, the tape is not stretchy.

The tape 30 may be a clear silicone and may be colored. The tape 30 may also be opaque and colored. The tape 30 may be a silicone-based tape or a wax tape, such as a clear, soft oral food grade silicon wax that covers a large area and attaches safely and more securely to keep the patient's cheeks safe. In addition, the tape 30 could also include whitening properties and/or additional functional benefits to breath and or pain prevention. The tape 30 can be used to lighten or whiten stains on brackets, teeth, clear aligner attachments, porcelain brackets, teeth, and discolored rubber bands. The tape 30 may also have flavors that are long lasting, such as for 12 hours, and may have patterns, designs, or other artwork to make them more interesting to the young patient.

The tape may be sold in rolls 32 that are positioned in dispensers 34, as shown in FIG. 1, compacts, strips 36 (as shown in FIGS. 2-4) and/or the like. The easy-to-use tape may be stored on a roll 32 and the patient may be permitted to cut off an amount of tape that is needed. This permits the patient to customize the tape 30 to their mouth size and to direct the tape 30 to specific areas of the mouth. The tape 30 may be stretchy so that the patient can pull it over the teeth and brackets to ensure it is secure.

The tape 30 can also include treatments to help the patient's mouth heal and/or feel or look better. For example, the tape 30 may include peppermint or other mint along with a whitening ingredient. Another example includes the use of licorice and lidocaine to sooth the pain from the braces. The tape 30 may include a wax-based material or may be non-wax-based. The tape 30 may be food grade silicone. Different varieties may include: 1) Breath freshener, 2) Antimicrobial ingredients, 3) Lidocaine for pain relief, 4) menthol or mint, or other combinations and ingredients.

In addition to the tape 30 working on its own, it can be used in conjunction with orthodontia mouth guards, as described below. The user places the tape on your teeth and braces before putting on the mouth guard to allow the mouthguard to slide on more easily. This protects the brackets from getting pulled and loosened and allows for the patient to have less pain when they put the mouthguard in and out.

In addition to providing pain relief, oral health care strips 30 for use with braces may also assist a patient in maintaining proper hygiene. For example, strips or tape 30 can be used to prevent bad breath and to prevent and/or treat discoloration of the teeth, among other challenges. Bracket strips 30 are typically of a pre-cut length and may be utilized to cover the teeth, brackets, and attachments during orthodontia treatment. The strips 30 are used to lighten or whiten stains on brackets, teeth, clear aligner brackets, teeth, and discolored rubber bands, especially with ceramic brackets. The strips 30 are designed to cover either only the front of the teeth or may be wide enough to cover the front and rear of the teeth by folding over the bottom edge of the teeth to cover both the front and back of the teeth. In addition, the oral health care strips 30 can include a breath freshener to both whiten and refresh at the same time. In addition, the oral health care strips 30 can include zinc, Vitamin D3, Vitamin C, a probiotic ingredient, and other ingredients, as desired.

The strips 30 may also include other ingredients for inhibiting activity of pathogenic bacteria with no toxicity. Some possible ingredients include S. Persica (Salvadora persica, which is a species of Salvadora, used for centuries as a natural toothbrush, also known as Miswak) and C. gileadensis (Commiphora gileadensis, the Arabian Balsam Tree, which is a shrub species), both of which are plant extracts. Other plant extracts and/or other ingredients may alternatively be used to reduce bacteria in the mouth. These ingredients may also be incorporated into other inventions and devices, including in gels, rinses, candy, and the like. The strips 30 may come in varied sizes depending on the number of teeth or size of mouth being treated.

In another embodiment of the oral health care strip, a fast-dissolving film is utilized to deliver ingredients and treatments to a patient. Fast-dissolving drug delivery systems have been developed as an alternative to conventional dosage forms as an oral means of drug delivery. Fast dissolving films are often preferred over conventional tablets and capsules because they can mask the taste of bitter drugs. This can result in an increase in patient compliance. Fast dissolving films are typically thin oral strips which dissolve in less than a minute when placed on the tongue. Currently known breath strips utilize this technology. These types of strips can also be used for delivery of vitamins, vaccines, and other drug products to the teeth, mouth, and gums.

In one embodiment, the comfort strip 30 may be a bio adhesive delivery system where there is a bond between a natural or synthetic polymer and the soft tissue epithelial cells in the mouth. The strips can either be positioned directly on the skin of the mouth or can be applied to the braces and teeth. Current oral strips dissolve on the tongue. Medicinal delivery via the oral transmucosal systems with an emphasis on the soft palate and the buccal mucosa may be used. The oral soft palate is a promising micro adhesive site for delivering active pharmaceuticals both systemically and locally. This bio adhesive delivery system will allow for immediate delivery and is a desirable mode of action for addressing orthodontic issues, including discomfort, halitosis, inflammation, demineralization, and extrinsic staining. The strips 30 may include vitamins, probiotics, and natural soothing ingredients to address the concerns of the orthodontic patient. The strips 30 may come in assorted flavors and may provide additional medicinal qualities as well as mode of transmission in the oral cavity.

The strips 30 may be double sided and may be made of a hydrogel 54, such as a poly vinyl pyrrolidone matrix that contains specified ingredients, such as natural anti-irritants. These ingredients may also include sorbitol and flavors like mocha, peppermint, among other flavors and ingredients. The strips 30 are designed to help relieve gum irritation and to help prevent mouth dryness. The strips 30 may provide a wet, low friction surface to sooth the vestibule. The strips 30 may also be used to reduce halitosis and to enhance the overall experience of wearing braces.

The strips 30 may be packaged as 12 strips or pieces per package. One possible size of the strip 30 is 40 mm×9.5 mm×1 mm. The package holding the strips 30 may be a molded flip-top case 38 that includes 12 individually wrapped strips. Alternatively, the case may have a sliding cover and a receptacle in which the strips rest (not shown).

Each strip 30 may be sealed in an individual package. One type of material that may be used to seal the package is a foil wrap. In a preferred embodiment, the strips 30 may be clear and/or translucent. The strips 30 may also have a long-lasting shelf life, such as at least 2 years.

As discussed above, polyvinyl pyrrolidone can be used in the strip. Polyvinyl pyrrolidone is a hydrogel 54. Other hydrogels 54 may also be used, including synthetic or naturally derived water-swellable gels. Examples of hydrogels 54 may comprise the polymers and their derivatives, and copolymers including, without limitation, polyacrylates, polyacrylate copolymers, polyvinyl alcohols, polyvinyl pyrrolidones, polyalkylethers, polysaccharides, and carbohydrates—in particular alkylated derivatives of cellulose. The hydrogel 54 may have a water content ranging from about 5% to about 50%. One type of hydrogel 54 that may be used is Comfort Gel A Part No. M807, made by R & D Medical Products, Inc., of Lake Forest, Calif. Comfort Gel A Part No. M807 has been found to meet the intended requirements of ISO 10993-10:2010.

The strips may be formed as layered products. The top layer is a polymer film 56, such as PE or PU. The next layer is a hydrogel 54 that is itself a formulation of water-water swellable polymer and excipients. The third layer is a release liner or carrier card 58, which is a polymer or silicone coated paper or polymer such as PP, PET, polyester, or card stock paper. The carrier card or release liner 58 does not contact a patient's mouth and is removed from the strip prior to use. The polymer film 56 adheres to and covers the hydrogel 54 but prevents adhesion to unintended surfaces in a patient's mouth. The hydrogel layer 54 is the material that adheres directly to surfaces in the mouth, whether it be the soft tissues, teeth and/or braces. The outer layer, or farthest removed layer from the adhesion surface inside the mouth, is the polymer layer 56. It has no adherence to the mouth, but it will have contact with tissue inside the mouth. A scrim material 60, such as a non-woven polyester fabric may be used to support the gel within the laminated system under the polymer film 56. The polymer film 56 is laminated onto the hydrogel coating 54 onto the release liner 58 and before die-cutting and packaging of the product. The layers of the strip are assembled using a lamination manufacturing process, as known by those of skill in the art.

Other ingredients may be added to the hydrogel 54, if desired. For example, flavorants may be mixed with the hydrogel 54 prior to the lamination process. In addition, a non-sugar sweetener can be added to the hydrogel 54 to make the strip taste sweet. One such sweetener is xylitol. Possible flavors include mint, menthol, dead sea salt, vanilla extract, blueberry, raspberry, winter mint, licorice root, mocha, mint, parsley, wasabi, ginger, probiotic L. salivarius WB21, among other flavors. Possible flavor combinations include: 1) mint, menthol, dead sea salt, vanilla extract, and xylitol; 2) blueberry flavor, raspberry flavor, and xylitol; 3) winter mint and licorice root; 4) mocha, mint, and parsley; and 5) wasabi, ginger, probiotic L. salivarius WB21, and xylitol. Other combinations are also readily conceivable.

The strips may be popular flavors, such as “vanilla sugar cookie,” which is infused with anti-inflammatory and anti-bacterial properties to fight gingivitis and cavities; “mint chocolate chip,” which is infused with mint extract and whitening agents to eliminate bad breath and remove extrinsic stains on enamel; and “caramel latte,” which is infused with pre/probiotics, vitamins and minerals promoting a healthy environment in the mouth.

Strips 30 may be formulated to meet certain needs. For example, strips 30 may be designed to clean and remineralize the teeth and may include as ingredients Dead Sea salt, coconut oil, xylitol, a probiotic, L. salivarius WB21, Ascorbic Acid, 3% Sodium Bicarbonate, 3% Calcium, 3% 2.5% Vitamin D, phosphorous, calcium carbonate, hydrated silica, zinc citrate, sodium bicarbonate, and/or sodium chloride. Strips 30 may be designed to treat halitosis and dry mouth and may include as ingredients 0.2% Green Tea Extract Monk Fruit 1%, Xanthan Gum 0.75%, Zinc Acetate 0.5%, Stevia 0.2%, Mountain Savory Essential Oil 0.125%, a probiotic, L. Salivarius WB21, a prebiotic, and/or xylitol. Strips 30 may be designed to treat pain and discomfort and may include as ingredients Dark chocolate, Wasabi, CBD, and/or ginger. Strips 30 may be designed to be anti-Microbial, anti-Bacterial, antioxidant, and to prevents cavities.

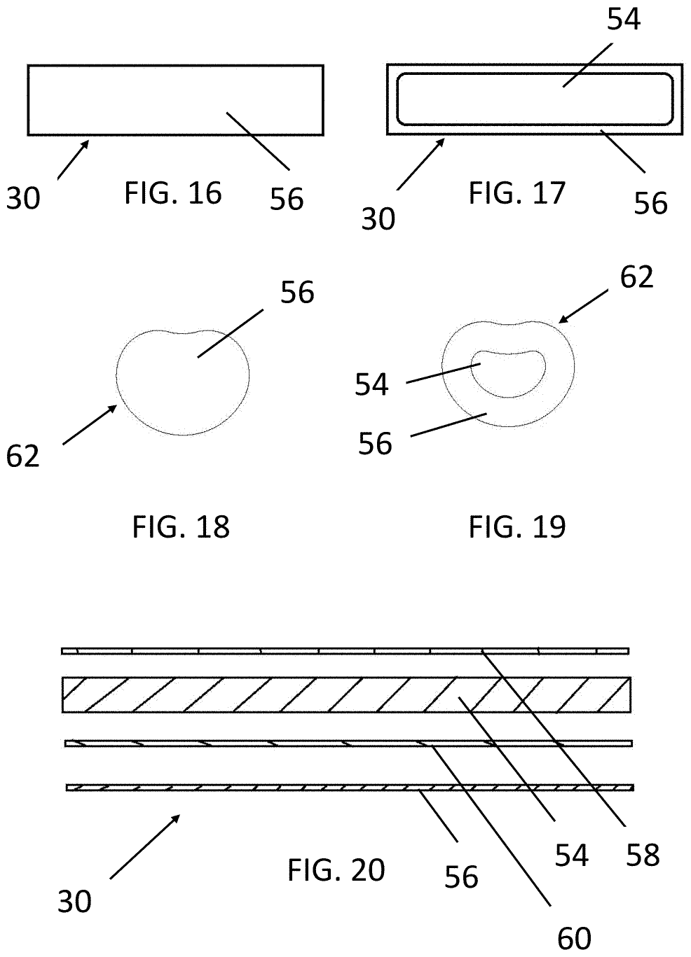

Referring to the figures, FIGS. 16-21 show various embodiments of the comfort tape 30, strips or therapeutic sticker 62 according to the invention. FIGS. 16-17 depict a comfort tape 30 that is rectangular shaped and that includes an inner hydrogel layer 54 on the bottom of the tape 30 that can be positioned against a user's teeth and/or braces. The top layer 56 of the tape 30 remains with the hydrogel 54 during use and a release liner 58, which is not shown in FIGS. 16-17 is positioned against the hydrogel layer 54 during storage. The top layer 56 may be a polymer film 56. The tape 30 is meant for self-application by the patient and is easy to use.

FIGS. 18-19 depicts a therapeutic sticker 62 that can be used on the soft tissue inside the mouth for improving oral health. The sticker 62, as shown, is semi-circular in shape, although other shapes may be used, such as circular, square, rectangular, triangular, or the like. The sticker 62 is like the tape shown in FIGS. 16-17 in that it includes a top polymer layer and a hydrogel positioned on the bottom layer during use in the mouth. A release liner 58 may also be positioned against the hydrogel during storage. The therapeutic sticker 62 has a shape and size that permits easy use in the mouth of a patient. The stickers 62 are meant for self-application by the patient and are easy to use.

In one embodiment, the sticker 62 may have a width of 32 mm, a height of 25.3 mm, and a thickness of 0.78 mm. The hydrogel 54 component on the sticker 62 may substantially match the exterior shape of the sticker 62, as shown, or could be different from the shape of the sticker 62. The width and height of the hydrogel layer 54 on the sticker 62 may be smaller than the width and height of the sticker 62. The width and height of the hydrogel 54 may be 18 mm by 11.3 mm, with a thickness of 0.7 mm. Alternatively, although not shown, the hydrogel layer 54 could cover the entire width and height of the sticker 62. The same is true for the tape, which may have a length and width for the hydrogel layer 54 that is smaller than the length and width of the tape. Alternatively, the length and width of the hydrogel layer 54 can be the same as the length and width of the tape. The tape and the sticker may be stored in a foil pouch. Alternatively, the tape and sticker can be stored in any type of packaging desired.

FIG. 20 depicts the various layers of the comfort tape 30 or therapeutic sticker 62 with a full thickness hydrogel layer. The various layers are laminated together to form the tape 30 or sticker 62. The lamination process may be performed in any way presently known or known in the future to create laminations. The layers include a lowermost release liner 58, a hydrogel 54, and a polymer layer 56. A non-woven scrim layer 60 may be positioned between the polymer layer 56 and the hydrogel 54 to stabilize the hydrogel 54 on the polymer layer 56. The polymer layer 56 may be a polyolefin film, for example.

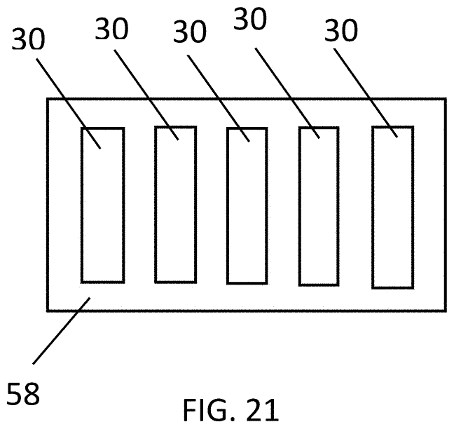

FIG. 21 depicts a carrier card 58/release liner 58 having multiple comfort tapes 30 adhered thereto. A patient would select a tape to position on their teeth and braces. Multiple strips could be used at a single time, if desired. The carrier card can be stored in a recloseable package so that a user can use any number of strips at a time and store the remainder.

In another embodiment, an orthodontic chew 10 is an elongated tubular member having a hollow interior 14. An opening 22 is provided in the elongated tubular member for admitting a liquid into the hollow interior 14. An exit 24 from the hollow interior 14 is provided that permits the liquid to exit the elongated tubular member 12. The hollow interior 14 may extend longitudinally. The liquid may have a thick consistency, like a gel of gelatin, so that the liquid flows through the hollow interior 14 slowly.

The elongated tubular member 12 may be non-edible, food grade silicone. The elongated tubular member 12 may have a hardness that permits a patient to repeatedly chew on the elongated tubular member 12 without damaging the elongated tubular member 12. The elongated tubular member 12 may be formed from two separate parts A, B that are fitted together. The liquid may include at least a flavorant.

All or some of the above-described items can be included as a set to be provided to patients for orthodontia care. The set may include some basics items that are needed for daily care of new braces and clear aligners, as well as a few samples of candy that has added health benefits. Starter kits may be provided that include a wire clipper, chocolates or chews 10, a tongue sponge, antimicrobial spray, and other products.

In one embodiment, an orthodontic chew 10 for use with braces includes an elongated tubular member 12 made of a chewable food grade silicone. The tubular member 12 includes an ingredient that improves oral health during usage of braces.

The ingredient may be included in the tubular member 12 by any known means, including coating, dosing, embedding, encircling, or other known means for including an ingredient in silicone. The elongated member 12 may have a length and a width. A cross-sectional shape of the elongated member 12 may be round. The elongated member 12 may be shaped in a curved shape. The elongated member 12 may be non-curved. The elongated tubular member 12 may have rounded ends.

The ingredient may be one or more of a flavoring, a numbing agent, a teeth cleaning agent, a whitening agent, and a breath freshening ingredient, as well as any other ingredient disclosed herein. The silicone may be a food grade siloxane polymer. The silicone may have a hardness of Shore 00-35. The elongated tubular member 12 may have a center matrix 14 that includes additional ingredients. The additional ingredients may include one or more of natural polysaccharide, pvp base, xylitol, sodium bicarbonate, menthol, spearmint oil, or flavoring, as well as any other ingredient disclosed herein. The elongated tubular member 12 may be either transparent or opaque. The elongated tubular member 12 may be either colored or uncolored. Other ingredients, including any of those discussed herein, may be included in the chew.

In another embodiment, a comfort tape or strip 30 for use with braces includes a strip of flexible and/or stretchable material that is infused with an ingredient to improve oral care during treatment with braces or aligners. The flexible and stretchable material may be a poly vinyl pyrrolidone matrix. The ingredient may be one or more of sorbitol, baking soda, an antimicrobial agent, a flavoring, a breath freshener, a whitening agent, and a pain reliever, among other ingredients disclosed herein. The tape 30 may be double sided. The tape 30 may be clear and/or translucent. The tape 30 may be sealed in packaging. The tape 30 may have a shelf life of two years or more. The tape 30 may be of a continuous length that is stored on a roll 32, or the tape 30 may be pre-cut 36 into desired lengths. The tape 30 may have adhesive properties. The tape 30 may provide a medicinal delivery system. The tape 30 may be a food grade silicone material. The tape may be multi-layered and may include multiple ingredients, such as those discussed above.

With braces, it is hard to floss. Most people find it impossible to floss properly. When patients do not floss, gums may hurt and sometimes may itch due to swelling. Longer term, failure to floss can cause periodontal disease and cavities.

A floss 40 specifically designed for use with orthodontia treatment is used to help patients keep fresh breath, clean teeth, and comfort. The floss 40 is bendable and provides a soft tip 42 that enables between teeth flossing. The system uses a spongey floss that can be fed between teeth to get into hard-to-reach areas. The floss 40 is designed specifically for ortho care as is made of a soft fiber thread that feels more like a sponge. Its soft fibers are soaked in a lidocaine, baking soda blend and antibacterial spray to help ease discomfort, to act as a super cleaning agent, and to improve overall oral care. Each floss strip may be precut or can be provided on a reel 44. The floss 40 may be formed of several materials. In one embodiment, the floss 40 is made of two materials: a non-latex rubber to hold its shape, similar to known gum pick products, covered by a softer, thinner material, such as a spongey material. One type of material that may be used, for example, is a hydrophilic foam, such as that used for makeup sponges, or other types of foam.

Another type of floss that may be used is a pick 46, as shown in FIGS. 14 and 15. The picks are curved and have a soft pointed tip tool 48 that can go through contact points between the teeth. The curved shape helps the tool to get into harder to reach areas behind braces. The picks are made of thin and soft plastic that can glide easily through the teeth. The floss picks are precut and sized to permit their entry between teeth.

The picks 46 include a semi-rigid material forming the lower part 50 of the pick 46 and a spongey material for the upper part 52 of the pick 46. The upper part 52 of the pick 46 is the portion that slides between the teeth first. The spongey material may be, for example, a hydrophilic foam, among other types of foam/sponge material. The floss may be coated or embedded with any of the ingredients discussed above, or other ingredients as desired.

Gels may also be provided to improve oral health. These gels may be used as toothpastes or as gels that can be rubbed on the gums. Possible combinations for the gels include:

1) Water 88.175%, Vegetable Glycerin 9.5%, Carbomer 1%, Xanthan Gum 0.5%, Zinc Acetate 0.5%, Stevia 0.2%, Cinnamon Oil Extract 0.125%, and Probiotics 45 million CFU's. Other additives may include mint oil extract, parsley extract, and cocoa extract. Microcrystals can be substituted for the cinnamon oil extract.

2) Water 86.5%, Vegetable Glycerin 9.5%, Carbomer 1.5%, Monk Fruit Extract 1%, Xanthan Gum 0.75%, Zinc Acetate 0.5%, Stevia 0.2%, Cinnamon Oil Extract 0.125%, Mountain Savory Essential Oil 0.125%, and Probiotics 45 million CFU's.

3) Water 85.5%, Vegetable Glycerin 9.5%, Carbomer 2%, Monk Fruit Extract 1%, Xanthan Gum 1%, Zinc Acetate 0.5%, Stevia 0.2%, Peppermint Oil Extract 0.125%, Labrador Essential Oil 0.125%, and Probiotics 45 million CFU's.

According to another embodiment of the invention, a tape for use with braces or aligners includes a member of flexible and stretchable material that is infused with an ingredient to improve oral care during treatment with braces or aligners. The material comprises two or more layers of material that are laminated together and that include a first layer of polymer film and a second layer of hydrogel material.

The polymer film of the first layer may be polyethylene or polyurethane. The hydrogel may be a water swellable polymer. The member may also include a third layer in the form of a release liner. The release liner may be a polypropylene, polyethylene terephthalate, or a card stock paper. The hydrogel may be one or more of polyacrylates, polyacrylate copolymers, polyvinyl alcohols, polyvinyl pyrrolidones, polyalkylethers, polysaccharides, and carbohydrates. The carbohydrates may be alkylated derivatives of cellulose.

The hydrogel may include water in a range from about 5% to about 50%. The hydrogel may be a synthetic or naturally derived water-swellable gel. The hydrogel may comprise polymers and their derivatives, or copolymers.

The flexible and stretchable material may be a poly vinyl pyrrolidone matrix. The flexible and stretchable material may be a polyacrylate or a polyacrylate copolymer.

The ingredient may be one or more of sorbitol, baking soda, an antimicrobial agent, a flavoring, a breath freshener, a whitening agent, and a pain reliever. The tape may have adhesive properties and may provide a medicinal delivery system. The tape may also include one or more of flavorants, plasticizers, antioxidants, adhesion promoters, anti-microbials, extracts, oils, sweeteners, probiotics, prebiotics, and salts. The tape may have a length ranging from about 0.1 to about 10 cm, a width ranging from about 0.1 cm to about 10 cm, and a thickness ranging from about 0.001 cm to about 0.1 cm. One possible size for the strip is about 3 cm long by 0.7 cm wide by 0.07 cm thick. Other sizes may alternatively be used. The hydrogel layer of the strips may be full thickness or could be island shaped.

According to another embodiment of the invention, a therapeutic sticker 62 for application to the soft tissue inside the mouth of a patient is a flexible member that is infused with an ingredient to improve oral care. The material comprises two or more layers of material that are laminated together and that include a first layer of polymer film and a second layer of hydrogel material. A therapeutic agent is associated with the hydrogel for transmission to the soft tissues of the mouth of a patient during use.

The flexible member may be configured for self-application by the patient. The hydrogel may be an acrylic polymer hydrogel. The hydrogel may also include at least one additive comprising one or more of flavorants, plasticizers, antioxidants, adhesion promoters, anti-microbials, extracts, oils, sweeteners, probiotics, prebiotics, and salts. The hydrogel may have adhesion properties that permit the flexible member to adhere to the soft tissues inside the mouth of a patient. The flexible member may be semi-circular in shape.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim. The term “consisting essentially,” if used herein, means the specified materials or steps and those that do not materially affect the basic and novel characteristics of the material or method. All percentages and averages are by weight unless the context indicates otherwise. If not specified above, the properties mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary.

What is claimed is:

1. A tape for use with braces or aligners comprising:
a member of flexible and stretchable material that is infused with an ingredient to improve oral care during treatment with braces or aligners, said material comprising two or more layers of material that are laminated together and that include a first layer of polymer film and a second layer of hydrogel material.

2. The tape of claim 1, wherein the polymer film of the first layer is polyethylene or polyurethane.

3. The tape of claim 1, wherein the hydrogel is a water swellable polymer.

4. The tape of claim 1, further comprising a third layer in the form of a release liner.

5. The tape of claim 4, wherein the release liner is a polypropylene, polyethlene terephthalate, or a card stock paper.

6. The tape of claim 1, where in the hydrogel is one or more of polyacrylates, polyacrylate copolymers, polyvinyl alcohols, polyvinyl pyrrolidones, polyalkylethers, polysaccharides, and carbohydrates.

7. The tape of claim 6, wherein the carbohydrates are alkylated derivatives of cellulose.

8. The tape of claim 1, wherein the hydrogel includes water in a range from about 5% to about 50%.

9. The tape of claim 1, wherein the hydrogel is a synthetic or naturally derived water-swellable gel.

10. The tape of claim 1, wherein the hydrogel comprises polymers and their derivatives, or copolymers.

11. The tape of claim 1, wherein the flexible and stretchable material is a poly vinyl pyrrolidone matrix.

12. The tape of claim 1, wherein the flexible and stretchable material is a polyacrylate or a polyacrylate copolymer.

13. The tape of claim 1, wherein the ingredient is one or more of sorbitol, baking soda, an antimicrobial agent, a flavoring, a breath freshener, a whitening agent, and a pain reliever.

14. The tape of claim 1, wherein the tape has adhesive properties and provides a medicinal delivery system.

15. The tape of claim 1, further comprising one or more of flavorants, plasticizers, antioxidants, adhesion promoters, anti-microbials, extracts, oils, sweeteners, probiotics, prebiotics, and salts.

16. The tape of claim 1, wherein the tape has a length ranging from about x to about x, a width ranging from about x to about x, and a thickness ranging from about x to about x.

17. A therapeutic sticker for application to the soft tissue inside the mouth of a patient comprising:
a flexible member that is infused with an ingredient to improve oral care, said material comprising two or more layers of material that are laminated together and that include a first layer of polymer film and a second layer of hydrogel material, with a therapeutic agent associated with the hydrogel for transmission to the soft tissues of the mouth of a patient during use.

18. The sticker of claim 17, wherein the flexible member is configured for self-application by the patient.

19. The sticker of claim 17, wherein the hydrogel is an acrylic polymer hydrogel and further comprising at least one additive comprising one or more of flavorants, plasticizers, antioxidants, adhesion promoters, anti-microbials, extracts, oils, sweeteners, probiotics, prebiotics, and salts.

19. The sticker of claim 17, wherein the hydrogel has adhesion properties that permit the flexible member to adhere to the soft tissues inside the mouth of a patient.

20. The sticker of claim 17, wherein the flexible member is semi-circular in shape.
